(19) United States
(12) Patent Application Publication
(10) Pub. No.: US 2006/0067917 A1
(43) Pub. Date: Mar. 30, 2006

(54) Insulin delivery system

(76) Inventors: Athanassios Sambanis, Atlanta, GA (US); Shing-Yi Cheng, Atlanta, GA (US)

Correspondence Address: MERCHANT & GOULD PC, P.O. BOX 2903, MINNEAPOLIS, MN, 55402-0903, US

(21) Appl. No.: 11/230,363

(22) Filed: Sep. 20, 2005

Related U.S. Application Data
(60) Provisional application No. 60/611,544, filed on Sep. 20, 2004.

(51) Int. Cl. A61K 35/39; A61K 38/16

(52) U.S. Cl. 424/93.7; 514/2

(57) ABSTRACT

Aspects of the disclosure generally relate to insulin delivery systems and compositions having a insulin secreting β cell line or insulin secreting recombinant non-β cells sequestered in a glucose-responsive material. The disclosed insulin delivery systems can be surgically implanted in a host and can provide a continuous source of insulin from the cells contained in the device. The combination of living cells with a glucose-responsive material provides a hybrid insulin delivery system that delivers physiologically relevant amounts of insulin in response to physiologically relevant glucose levels. In some aspects, the disclosed devices have a biphasic release of insulin in response to sudden increases in glucose concentrations in the fluids bathing the device.

### CROSS-REFERENCE TO RELATED APPLICATION

This application claims priority to and benefit of U.S. Provisional Patent Application No. 60/611,544 filed on Sep. 20, 2004, and which is incorporated by reference in its entirety.

### STATEMENT REGARDING FEDERALLY SPONSORED RESEARCH OR DEVELOPMENT

Aspects of the disclosed work were supported in part by the National Science Foundation under award number EEC-9731643 and by the National Institutes of Health award number DK 56980. Therefore, the US government has certain rights in the disclosed subject matter.

### BACKGROUND

1. Technical Field

The disclosed subject matter is generally related to compositions, devices, and systems for regulating physiological responses in a host, in particular medical devices comprising living cells for regulating glucose or insulin levels in a host.

2. Related Art

Insulin-dependent diabetes is a serious disease affecting more than 3 million people in the US. The current treatment commonly consists of frequent blood glucose monitoring and multiple daily insulin injections. However, this treatment does not provide the tight glycemic regulation afforded by a normally functioning pancreas, thus it cannot prevent long-term complications, which include cardiovascular disease, retinopathy and nephropathy. Closed-loop insulin delivery systems, consisting of a glucose sensor, a controlled pump, and an insulin reservoir, are promising at reducing the occurrences of hypo- and hyperglycemia. However, the clinical application of such systems is still held back by technical difficulties, primarily the in vivo longevity and stability of glucose sensors.

Cell-based therapies have significant potential in correcting the drawbacks of the insulin delivery systems mentioned above. Transplantation of human islets from cadaveric donors following the Edmonton protocol, which uses steroid-free immunosuppressive drugs, results in insulin independence for 80% of the treated patients over a period of 12 months. However, as the number of available donors will never meet the large number of patients, and the risk of life-long immunosuppression is of concern, immunologically acceptable insulin-producing cells that exhibit proper secretion dynamics need to be sought. Non-β cells, such as fibroblasts, myoblasts, and hepatocytes, can be retrieved as a biopsy from the same patient and engineered to express recombinant insulin. Due to their autologous nature, such cells relax the immune acceptance challenges, however, their insulin secretion is either constitutive or regulated at the transcriptional level, thus constant or sluggish in response to physiologic stimuli and inadequate for glycemic regulation in higher animals and humans.

Accordingly, improved systems and devices for regulating glucose or delivering insulin are needed.

### SUMMARY

Aspects of the disclosure generally relate to insulin delivery systems and compositions having a insulin secreting β cell line or insulin secreting recombinant non-β cells sequestered in a glucose-responsive material. The disclosed insulin delivery systems can be surgically implanted in a host and can provide a continuous source of insulin from the cells contained in the device. The combination of living cells with a glucose-responsive material provides a hybrid insulin delivery system that delivers physiologically relevant amounts of insulin in response to physiologically relevant glucose levels. In some aspects, the disclosed devices have a biphasic release of insulin in response to sudden increases in glucose concentrations in the fluids bathing the device.

Other aspects provide methods of manufacturing the disclosed insulin delivery devices as well as methods of their use. One aspect provides a method for delivering insulin to host using the disclosed systems and compositions. Another aspect provides a method for treating diabetes by implanting the disclosed device into a host diagnosed with diabetes or suspected of having diabetes. Still other aspects provide methods of regulating carbohydrate metabolism of a host using the disclosed systems and compositions.

Yet another aspect provides a glucose-responsive hydrogel that becomes a sol when in contact with fluid having a glucose concentration of about 25 mM or more, typically of about 27.5 mM or more. The hydrogel can contain pegylated concanavalin A in a ratio of about 2.5 to about 5 PEG:con A. The hydrogel can also include glycogen.

Other aspects and uses will become apparent in view of the figures and examples provided herein.

### BRIEF DESCRIPTION OF THE FIGURES

Various embodiments are illustrated in the drawings in which like reference characters designate the same or similar parts throughout the figures. The figures are not drawn to scale.

FIG. 1 shows a representative insulin delivery system.

FIGS. 2A and 2B show plasmid structures CMV/Furin-B10 PPI cDNA/Puro^{R} and CMV/Furin-B 10 PPI cDNA, respectively.

FIG. 3 shows insulin release profiles from hybrid construct of alginate-encapsulated insulin-secreting βTC3 cells with con A-based material (experiment, ▪) and with culture medium (control, □). In each independent experiment, insulin release rates were normalized to the rate during 0-2 hour, which was set equal to 1. ♦, *, #, and ▴ indicate pair-wise statistical comparisons using a one-tailed t-test, p<0.05. Error bars indicate standard deviation. N=3 for all.

FIGS. 4A and 4B show representative histology cross-sections of alginate-encapsulated HepG2 cells on days 3 and 6 post-encapsulation. Cells were encapsulated at an initial density of 7×10⁷ cells/ml of alginate in 700 μm diameter beads.

FIG. 5 shows insulin release profiles from hybrid construct of alginate-encapsulated insulin-secreting HepG2 cells with con A-based material (experiment, ▪) and with alginate (control, □). In each independent experiment, insulin release rates were normalized to the rate during 0-2 hour, which was set equal to 1. ♦, *, #, and ▴ indicate pair-wise statistical comparisons using a one-tailed t-test, p<0.05. Error bars indicate standard deviation. N=4 for all.

FIGS. 6A-C show representative histology cross-sections of alginate encapsulated insulin-secreting C2C12 cells on days 2 and 7 for cultures in medium with 10% FBS and on day 7 for cultures in medium with 2.5% HS. Cells were encapsulated at an initial density of 7×1 7 cells/ml of alginate in 700 μm diameter beads.

FIG. 7 shows insulin release profiles from hybrid construct of alginate-encapsulated insulin-secreting C2C12 cells with con A-based material (experiment, ▪) and with alginate (control, □). Media used were supplemented with 2.5% HS. In each independent experiment, insulin release rates were normalized to the rate during 0-2 hour, which was set equal to 1. ♦, *, #, and ▴ indicate pair-wise statistical comparisons using a one-tailed t-test, p<0.05. Error bars indicate standard deviation. N=3 for all.

FIG. 8 shows insulin release profiles from hybrid construct of alginate-encapsulated insulin-secreting C2C12 cells with con A-based material (experiment, ▪) and with alginate (control, □). Media used were supplemented with 10% FBS. In each independent experiment, insulin release rates were normalized to the rate during 0-2 hour, which was set equal to 1. ♦, *, #, and ▴ indicate pair-wise statistical comparisons using a one-tailed t-test, p<0.05. Error bars indicate standard deviation. N=3 for all.

FIG. 9 shows viabilities of alginate-encapsulated cells at the end of construct experiments with con A-based material (experiment, ▪) and with culture medium (PTC 3 cells (n=3)) or alginate (HepG2 (n=4) and C2C12 (n=3) cells) (control, □). * indicate pair-wise statistical comparisons using a one-tailed t-test, p<0.03. Error bars indicate standard deviation.

FIGS. 10A and 10B show exemplary con A-based glucose-responsive materials before and 30 minutes after exposing to glucose solutions, respectively.

FIG. 11 shows amounts of con A molecules detected at the outside of the 0.02-μm pore size membrane tissue-culture inserts containing different types of con A-based material (PEG 5-con A-glycogen (black bar), PEG 2.5-con A-glycogen (gray bar), and con A-glycogen (white bar)) 6 hours after exposing to glucose solutions (n=3). #* and + indicate pair-wise statistical comparisons using a one-tailed t-test, p<0.05. Error bars indicate standard deviation.

FIG. 12 shows FITC-Insulin-release rates from hybrid constructs containing different types of con A-based material (PEG 5-con A-glycogen (black bar), PEG 2.5-con A-glycogen (gray bar), and con A-glycogen (white bar)). In each independent experiments, FITC-insulin release rates obtained in the high glucose solution (concentration indicated at the x-axis) were normalized to the rates obtained in the glucose-free solution, which was set equal to 1 (n=3). #, *, ♦, +, [symbol] , and § indicate pair-wise statistical comparisons using a one-tailed t-test, p<0.03. Error bars indicate standard deviation.

FIGS. 13A-C show typical insulin-release profiles from hybrid constructs of alginate-encapsulated insulin-secreting C2C12 cells with PEG 5-con A-glycogen (A), alginate (B), and con A-glycogen (C).

FIG. 14 shows insulin-release profiles from hybrid constructs of alginate-encapsulated insulin-secreting C2C12 cells with PEG 5-con A-glycogen (filled bar), alginate (dashed bar), and con A-glycogen (blank bar). In each independent experiments, insulin release rates were normalized to the rate during 0-2 hours, which was set equal to 1 (n=4 for the experimental constructs containing PEG5-con A and n=2 for the control constructs containing alginate or unmodified con A material). *, #, §, and [symbol] indicate pair-wise statistical comparisons using a one-tailed t-test, p<0.05. Error bars indicate standard deviation.

### DETAILED DESCRIPTION

### 1. Definitions

In describing and claiming the disclosed subject matter, the following terminology will be used in accordance with the definitions set forth below.

The term “glucose-responsive material” refers to a material that changes permeability to insulin in response to changes in glucose concentration of fluid in contact with the material.

“Identity,” as known in the art, is a relationship between two or more polypeptide or polynucleotide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H, and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The term “insulin” refers to a polypeptide hormone that regulates carbohydrate metabolism. The term encompasses proinsulin, propreinsulin, insulin-like polypeptides, or other forms or variants of insulin that can be processed, for example, enzymatically, to produce a biologically active form or variant of insulin. Active forms of insulin include polypeptides that activate the insulin receptor directly or indirectly.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “treating” includes alleviating the symptoms associated with a specific disorder or condition and/or preventing or eliminating said symptoms.

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0+1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, the term “vector” is used in reference to a vehicle used to introduce a nucleic acid sequence into a cell. A viral vector is virus that has been modified to allow recombinant DNA sequences to be introduced into host cells or cell organelles.

### 2. Representative Embodiments

Insulin Delivery Systems

Insulin delivery systems, devices, compositions, and methods of their use are provided. Embodiments of the present disclosure provide an insulin delivery system comprising an insulin secreting β cell line or insulin secreting recombinant non-β cells sequestered in a glucose-responsive material. Certain embodiments provide an insulin delivery system that exhibits a biphasic secretory response similar to islet cells. A rapid first phase of insulin secretion results from the release of pre-synthesized insulin, and a second, more prolonged phase of secretion is due to insulin biosynthesis.

One embodiment provides an insulin delivery system incorporating a glucose-responsive material in combination with a continuous or renewable source of insulin. An exemplary glucose-responsive material includes, but is not limited to a concanavalin A (con A)-based glucose-responsive material. The continuous or renewable source of insulin includes one or more insulin producing cells. For example, mouse insulinoma βTC3 cells can be used as well as cells genetically engineered for constitutive insulin secretion. Representative recombinant cells that are engineered to constitutively secrete insulin include human HepG2 hepatomas or murine C2C12 myoblasts transfected with a nucleic acid encoding insulin or an insulin precursor. The glucose-responsive material and the insulin producing cells are discussed in more detailed below.

In one embodiment, release of the insulin from the device is regulated by the glucose-responsive material. Suitable glucose-responsive material is in a gel state at low glucose concentrations or about 5 mM glucose. In some embodiments, the glucose-responsive material with unmodified con A-glycogen gel had a low or gel state at about 25 mM, and a high or sol state at about 220 mM; PEGylated con A-glycogen had a low or gel state at about 5 mM, and a high or sol state at about 27.5 mM. When in the gel state, the material exhibits low insulin permeability. Generally, insulin permeability of the material under high glucose concentration (sol state) is approximately 3.5 times or more (based on the experimental conditions) higher than the permeability under low glucose concentration (gel state). Suitable insulin permeability for the glucose-responsive material is approximately 2.1×10⁻⁶ cm/s at gel state and 7.4×10⁻⁵ cm/s at sol state.

At high glucose levels, for example 25 mM or more, the material becomes a sol that exhibits a higher permeability to insulin. Thus, at low glucose concentrations, a higher fraction of the insulin secreted by cells accumulates within the construct, and the insulin release rate is relatively low. When the construct is exposed to high glucose, the material becomes a more permeable sol, and the insulin release rate from the construct is higher. As the time constant of the gel to sol and of sol to gel transformations is shorter than the (infinite) time constant of constitutively secreting cells or the (long) time constant of transcriptionally regulated cells, the kinetics of material transformation determine the kinetics of insulin release from the entire device.

In other embodiments, release of insulin from the device is controlled in part by regulating the biosynthesis of insulin or insulin like polypeptides from the cells in the device. For example, cells can be transfected with a promoter sequence responsive to levels of insulin so that at high levels of insulin, transcription of new insulin is retarded or abrogated in the transfected cells. An exemplary promoter that can be used includes the promoter developed by Thule et al (Gene Therapy, v7, p205-214, 2000), which has a glucose-stimulatory element (GIRE) from the L-type pyruvate Kinase L-PK promoter inserted directly upstream of an insulin-suppressive basal promoter of the rat insulin-like growth factor binding protein-1 (rIGFBP-1). Insulin secretion from the hepatocytes transfected with this promoter is stimulated by high glucose (5 to 25 mM) and inhibited by high insulin (10-10 to 10-7 M)

At low levels of insulin, the transfected cells will transcribe nucleic acids encoding insulin or insulin-like polypeptides. In certain embodiments, regulation of insulin transcription is combined with glucose-responsive material so that the insulin delivery device releases insulin in physiological amounts in response to mammalian physiological levels of glucose.

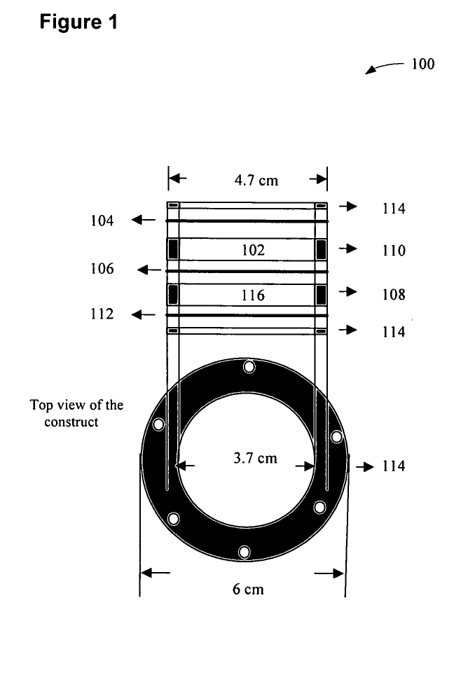

FIG. 1 shows a representative insulin delivery device 100 according to one embodiment of the disclosure. Although described as a disc, it will be appreciated that the device can be in any geometrical configuration. The glucose-responsive material can be a concanavalin-A (con A) based material, for example con A-glycogen material 102. The con A-glycogen material 102 is placed in a reservoir 110, for example a 3.7 cm diameter hole of a 2 mm-thick silicon sheet and is sandwiched between a 0.1 μm pore-size polycarbonate 104 and a 0.02 μm pore-size Anodisc membrane 106. Both of these membranes are permeable to insulin. To minimize leakage of con A towards the cell compartment while not excessively restricting diffusion of insulin through the membranes themselves, the smaller pore size membrane was used to separate the cells and the material. The larger pore size membrane was placed between the material and the surrounding medium.

The cell compartment 108 houses the insulin-producing cells 116 which are optionally alginate-encapsulated. Cell compartment 108 is bounded on one side by the material compartment 110 and on the other side by a 3 k Dalton MWCO polyethersulfone membrane 112 which allowed passage of small molecules, such as glucose and oxygen, for cellular nourishment, but effectively excludes insulin. Polycarbonate plates 114 with 6 screws were used to secure the components. The entire construct thickness was 1.0 cm.

In experimental constructs with glucose-responsive material, pieces of con A-glycogen gel of approximately 2 ml total volume were transferred by a spatula and used to fill the hole of the silicon sheet on top of the Anodisc membrane. Then, the polycarbonate membrane was positioned on top of the material to complete the material barrier. Two types of control constructs were fabricated, one with calcium alginate and the other with culture medium in place of con A-glycogen. In the alginate control, the material compartment was first assembled with the Anodisc and polycarbonate membranes and the polycarbonate plates sandwiching the silicon sheet tightly. Sodium alginate (ISP Inc.), filter-sterilized through 0.2 μm syringe filter (Pall Life Sciences), was then injected, filling the space in between the membranes. The whole construct was immersed in a 1.1% CaCl₂ solution for 30 minutes to ensure gelation of the alginate inside the barrier. For the control construct with culture medium, the medium was injected into the material compartment after the entire construct was assembled.

To assemble the cell compartment, 1 ml of alginate-encapsulated insulin-secreting cells were first placed on the top of the 3 kDa MWCO cellulose ester membrane on the lower polycarbonate plate. The material sandwich assembled as described previously was then laid on top of the cell compartment, followed by the material-side polycarbonate plate, and the whole construct was tightly assembled by screws. Approximately 1 ml of the culture medium was then injected into the cell compartment. The assembled construct was immediately immersed in a beaker containing 50 ml of culture medium with 25 mM glucose and supplemented with serum, as described in the cell culture section for the different cell lines. The beaker and the construct were then placed inside a tissue culture incubator on top of an orbital shaker that provided adequate mixing.

Glucose-Responsive Material

Glucose-responsive materials are known in the art, and have been used in developing glucose sensors and in controlling the release rate of insulin from drug delivery devices. However, in the latter, loss of insulin activity with time and the need to periodically refill the reservoir are problematic. Various types of glucose-responsive material have been proposed. For use in a hybrid device with cells, an ideal glucose-responsive material should exhibit the following characteristics: it should function under physiologic conditions, including pH, temperature, and physiologically relevant glucose concentrations; release no toxic compounds harming the cells; function with the form of insulin released by the cells, i.e., does not require modified insulin; exhibit high glucose specificity, rapid kinetics, and long-term stability.

Certain embodiments of the disclosed insulin delivery devices use a con A based glucose responsive material. Con A is a lectin which forms a tetramer under physiologic conditions and has four binding sites toward free glucose or glucose residues in a polysaccharide chain. When con A is mixed with polysaccharides, it acts as a crosslinker forming a highly viscous gel. When exposed to a high glucose solution, the gel dissociates into a sol due to the competitive binding reaction between free glucose and the glucose monomers in a polysaccharide chain toward con A.

An exemplary glucose-responsive material was produced by dissolving glycogen (Sigma Chemical Co., St. Louis, Mo.) in PBS at 60% (w/v) and mixing it with a 0.2M NaIO₄ solution at 1:1 volume ratio for 24 hours in the dark. The hydrogel was formed by mixing the resultant glycogen solution with a 15% w/v con A (Amersham Biosciences, Piscataway, N.J.) solution in PBS at a 1:1 volume ratio. The gel was left at room temperature 24 hours followed by 1 hour immersion in 1 mg/ml NaBH₄ at 4° C. Finally, the gel was washed at least three times with PBS buffer and stored in PBS at 4° C. for later use.

It will be appreciated that any glucose responsive material can be utilized that increases its insulin permeability as glucose levels of fluids in contact with the material increase. When exposing a con A-glycogen hydrogel to a glucose solution, the binding states of the con A molecules can be described in the following equations:

$$\begin{matrix}[ con A ] + [ glycogen ] ↔ [ con A \text{:} glycogen ] & ( 1 ) \\ [ con A ] + [ glucose ] ↔ [ con A \text{:} glucose ] & \\ Binding constant of {K}_{con A \text{:} glycogen} = \frac{[ con A \text{:} glycogen ]}{[ con A ] [ glycogen ]} & \\ [ con A \text{:} glycogen ] = {K}_{con A \text{:} glycogen} [ con A ] [ glycogen ] & \\ Binding constant of {K}_{con A \text{:} glycogen} = \frac{[ con A \text{:} glycogen ]}{[ con A ] [ glycogen ]} & ( 2 ) \\ [ con A \text{:} glycogen ] = {K}_{con A \text{:} glycogen} [ con A ] [ glycogen ] & \\ \text{Dividing equation (1) by (2):} & \\ \frac{[ con A \text{:} glucose ]}{[ con A \text{:} glycogen ]} = \frac{{K}_{con A \text{:} glucose} [ glucose ]}{{K}_{con A \text{:} glycogen} [ glycogen ]} & ( 3 )\end{matrix}$$

To have the material in the sol state, most of the con A molecules have to bind to the free glucose, in other words, [con A: glucose] has to be larger than [con A: glycogen]. As shown in equation (3), such condition can be achieved by increasing the concentration of free glucose. However, due to multivalency effects, con A generally has higher affinity towards polysaccharides than towards free glucose (e.g. K_{conA:dextran}=1.5×10⁴. M⁻¹ and K_{conA:glucose}=320 M⁻¹), hence, the glucose concentration required to induce the con A-glycogen gel into a sol is high beyond physiological range. One potential solution in improving the glucose sensitivity of the con A-based glucose-responsive material is by adjusting the binding constants, more specifically, decreasing the K_{conA:glycogen} and increasing K_{con A: glucose}.

Suitable glucose-responsive materials that can be used with the disclosed devices can be selected form at least three types of glucose responsive-materials reported in the literature. The first type, based on glucose oxidase, has received significant attention in drug delivery and the development of glucose sensors (J Biomed Mater Res 19, p1117-33, 1985; J Control Release 67, p9-17, 2000; Biomaterials 21, p1439-50, 2000; Biomaterials 21, p1679-87, 2000.) Glucose oxidase catalyzes the conversion of glucose to gluconic acid and hydrogen peroxide, thus lowering the pH when this reaction occurs. A hydrogel made from glucose oxidase and pH sensitive material may thus control the release of insulin by changing the structure of the pH-sensitive material when the reaction with glucose occurs.

A second type of material is based on phenylboronic acid, which can form a stable hydrogel with a polyol, such as poly(vinyl alcohol) (Pharm Res 14, p289-293 1997; Polymer Preprints 35, p393-394, 1994; J. Am. Chem. Soc. 120, p12694-12695, 1998.). This hydrogel is glucose-responsive, as it becomes dissociated in the presence of a competing polyol compound, such as glucose.

The third type of glucose-responsive material is based on the glucose-binding protein con A, which is obtained from the jack bean plant. Con A generally binds to saccharides containing a-D-mannose or a-D-glucose residues and appears to recognize terminal as well as internal saccharide residues. Con A exists as tetramers under neutral pH, with the tetrameric complex having four binding sites towards glucose. When mixed with polysaccharides such as glycogen (J Pharm Pharmacol 51, p1093-8, 1999), dextrans (Pharm. Pharmacol. Commun. 4, p117-122, 1998), and other types of synthetic polysaccharides (Biomaterials 18, p801-6, 1997; J Biomater Sci Polym Ed 6, p79-90, 1994, Journal of Controlled Release 77, p39-47, 2001), con A acts as a crosslinker forming a highly viscous gel. Free glucose in the surrounding solution can competitively displace the polysaccharide and dissociate the gel into a sol.

The con A-based material offers distinct advantages relative to the other glucose-responsive hydrogels for the following reasons: it is highly specific to glucose; it can function under physiologic environment; the kinetics of phase changes are relatively fast; the phase change is a reversible process; and it is not necessary to modify the insulin. Although con A is known to be immunogenic, the large molecular weight of the monomer (27,000) and especially of the tetramer that forms at neutral pH (108,000) enables the retention of the material in membranes, thus improving its biocompatibility.

One embodiment provides a glucose-responsive hydrogel. The hydrogel comprises pegylated con A and glycogen. In certain aspects, the PEG:con A ratio is about 2.5 to about 5. At such ratios, the hydrogel becomes a sol when in contact with glucose concentrations of about 25 mM or more, typically about 27.5 mM glucose or more. In other embodiments, the glucose-responsive hydrogel is a gel when in contact with fluids having a glucose concentration of about 5 mM or less. The hydrogel's permeability to insulin increases as the hydrogel shifts towards a sol in response to increase glucose levels. Shifting to a sol allows insulin in the hydrogel to be delivered to the fluid having glucose concentrations of more than about 5 mM.

In one embodiment, con A can be tethered to polymer chains. For example, con A can be modified with PEG using Acrl-PEG-NHS (Molecular weight of 3,400, Nektar Therapeutic, Huntsville, Ala.), which has two functional groups: the NHS group (N-hydroxylsuccinimide) is reactive towards lysine groups and is widely used for protein PEGylation, and the Acrl (acrylate) group offers the possibility of vinyl polymerization, which can be used to tether con A to polymer chains.

Using bifunctional PEG molecules to modify con A can achieve the following objectives: (1) improve the glucose sensitivity of con A-based material towards physiologic glucose concentrations; (2) improve the biocompatibility of con A molecules since PEGylation is a well established tool in reducing the immunogenicity of proteins; (3) enhance the stability (long-term maintenance of glucose responsiveness) of con A-based material by tethering con A molecules through PEG molecules to polysaccharides.

Insulin Producing Cells

Exemplary insulin delivery devices include a continuously refilled insulin reservoir. The insulin reservoir is continuously filled by insulin-secreting cells. Insulin can accumulate in the glucose responsive material when glucose levels are low in fluids surrounding or bathing the device. As glucose levels increase in surrounding fluids, the glucose responsive material becomes correspondingly more permeable to insulin thereby releasing more insulin into the surrounding fluids.

Typically, the device includes non-β cells that have been genetically engineered to secrete insulin or insulin-like polypeptides. Using non-β cells in the device advantageously avoids any autoimmune response the host may have to β-cells. Cells are typically harvested from a host, engineered to express insulin, and incorporated into the device to be implanted into that host. Suitable cells include cells that are capable of secreting polypeptides and include, but are not limited to myoblasts, hepatocytes, bone marrow, adipose tissue, fibroblasts, and combinations thereof. Exemplary cells and constructs that can be used with the disclosed device include those described in U.S. Patent Publication No. 20040142884 and International Patent Application Publication No. WO2005037226 both of which are incorporate by reference in their entirety.

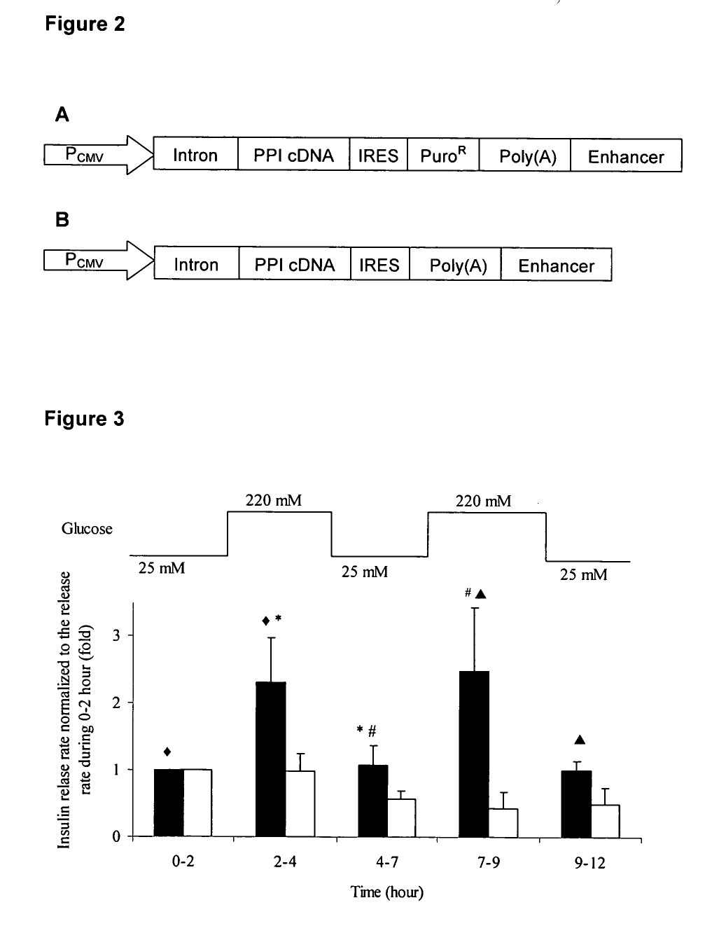

Suitable cells that can be used is with the disclosed devices, include but are not limted to hepatocytes, myoblasts, embryonic stem cells, adult stem cells, adipocytes, bone marrow cells, umbilical chord blood cells, and fibroblasts genetically engineered to become insulin-secreting cells. However, since most of the non-β cells do not carry the two prohormone convertases PC1/3 and PC2, which cleave the proinsulin into mature insulin, a Furin-cleavable human preproinsulin cDNA instead of a wild-type human preproinsulin DNA was used. This human preproinsulin cDNA was mutated at the B/C and C/A junctions so it can be processed into an insulin-like molecule in most cells by a ubiquitously expressed endoprotease, furin. The human preproinsulin cDNA (PPI cDNA) used in FIGS. 2A and 2B is one of several constructs that can be used. Similar gene elements that can be used include: INS/Fur. (Biochem Biophys Res Commun 265, p361-5 1999); FurHPI. (Hum Gene Ther 10, p1753-62 1999); hppI4 (J Endocrinol 172, p653-72 2002); p3MTChins (Gene Ther 12, p655-67 2005); hIns-M3 (Gene Ther 9, p963-71 2002).

Other than the use of a Furin-cleavable human preproinsulin cDNA, one can also use a single-chain insulin analogue developed by Lee et al (Nature 408, p483-8, 2000) in the plasmid, although this recombinant modified insulin has only 20-40% activity of human insulin.

Promoters such as a CMV promoter can be used so the engineered cells became constitutively insulin-secreting cells. Alternatively, other types of promoters such as the SV40 promoter developed by Thule et al (Gene Therapy 7, p205-214, 2000) can be used which is stimulated by glucose and inhibited by insulin, in such case, the insulin secreted by the cells would not over accumulate inside the cell compartment.

Still another embodiment provides transfecting a cell of the disclosed device with one or more therapeutic polypeptides. For example, a recombinant cell can be engineered to secrete insulin, proinsulin, preproinsulin, and a second polypeptide, for example a cytokine, chemokine, peptide hormone, growth factor, or the like. An exemplary second therapeutic polypeptide includes but is not limited to erythropoietin or thrombopoietin. The cells of the device can also be engineered to secrete fusion polypeptides comprising insulin or a fragment thereof in combination with a second therapeutic polypeptide.

Methods of Use

One embodiment provides a method for regulating glucose levels in a host by implanting one or more of the disclosed devices into a host. The device can be surgically implanted into a host so that the device is in fluid communication with the host's circulatory system. As the host's glucose levels increase, the device can release insulin into the circulatory system of the host to help regulate glucose levels.

Another embodiment provides a method for treating diabetes type I or II using the compositions and devices disclosed herein. A host having or suspected of having diabetes can be treated using one or more of the disclosed devices to deliver physiological amounts of insulin in response to the host's plasma or blood glucose levels.

Still another embodiment provides a method for regulating carbohydrate metabolism using the disclosed compositions and devices to deliver insulin or insulin-like polypeptides to a host in need thereof.

### EXAMPLES

### Example 1

### Cell Culture and Transfection

Autologous non-β cells can be retrieved as a biopsy from a patient. Among non-β cells, hepatocytes and myoblasts are particularly suitable for use as β-cell surrogates. Hepatocytes can be obtained as a biopsy, they express GLUT2 and glucokinase, two major glucose-sensing components in β cells that are involved in regulating insulin secretion, and hepatic expression of insulin under transcriptional regulation has been repeatedly shown to provide some glycemic control in diabetic rodents. However, due to the absence of an acute secretory response, it is unclear that engineered hepatocytes will provide appropriate glycemic regulation in higher animals, without the risk of hypoglycemic episodes. Myoblasts also lack a regulated secretion pathway, but relative to hepatocytes they offer the advantage of easier retrievability as a biopsy, capability in amplifying into large cell number, and the ability for differentiation into stable myotubes. Moreover, myoblasts have been engineered to constitutively secrete a number of therapeutic proteins, such as ciliary neurotrophic factor, erythropoietin, human factor IX, and human growth hormone. Prior to implantation, myoblasts can be differentiated into myotubes after encapsulation inside polyethersulfone hollow fibers, or they can be tissue-engineered in vitro into a bioartificial muscle.

βTC3 cells were obtained from the laboratory of Shimon Efrat, Albert Einstein College of Medicine, Bronx, N.Y. Cells were cultured in Dulbecco's modified Eagle's medium (DMEM, Sigma) supplemented with 15% horse serum (Sigma), 2.5% fetal bovine serum (Hyclone, Logan, UT), 1% penicillin/streptomycin (Mediatech, Herndon, Va.), and L-glutamine (Gibco, Grand Island, N.Y.) to a final concentration of 6 mM. HepG2 human hepatoma cells (ATCC, Manassas, Va., USA) and C2C12 mouse skeletal myoblasts (ATCC) were cultured in DMEM (Mediatech) supplemented with 10% fetal bovine serum (Hyclone) and 1% penicillin/streptomycin (Mediatech). C2C12 myoblasts were induced to differentiate into myotubes by culturing in low serum medium consisting of DMEM (Mediatech) supplemented with 2.5% hourse serum (Hyclone) and 1% penicillin/streptomycin (Mediatech). Myotube formation was observed under the microscope 3 days after the cells were switched to the low serum medium. C2C12 cells were transfected as myoblasts.

The plasmid, CMV/Furin-B10 PPI cDNA/Puro^{R}, used to engineer HepG2 and C2C12 cells into stable insulin-secreting cells, was constructed as shown in FIG. 2A. Briefly, a DNA fragment containing IRES and puromycin resistance genes from plasmid pIRESpuro (Clontech, Palo Alto, Calif.) was connected to the 3′-end of the Human preproinsulin (PPI) cDNA in the vector, CMV/Furin-B10 PPI cDNA (FIG. 2B). The PPI cDNA was furin-compatible with a His B10-to Asp mutation (B10 mutation), and was a kind gift of Genetech, San Francisco, Calif. The expression cassette of plasmid CMV/Furin-B10 PPI cDNA/Puro^{R} generated a bicistronic mRNA and directed the synthesis of both the insulin and puromycin resistance protein, thus facilitating the selection process of stable colonies.

When HepG2 and C2C12 cells reached about 60% confluency, they were treansfected using the FUGENE 6 reagent (Roche, Indianapolis, Ind.) according to the manufacturer's protocol. In a T-75 flask, cells were incubated 24 hours with the culture medium (without antibiotics) containing the plasmid (15 μg) and the FUGENE 6 reagent (45 μl). After transfection, cells continued to be cultured for recovery until they were ready to be split. When the transfected cells reached about 90% confluency, cells were trypsinized and split in 1:100 ratios into 100 mm culture dishes. During the selection process, which lasted over one month, cells were cultured in the presence of 2 μg/ml puromycin. Only cells that expressed the puromycin resistance gene survived under the selection pressure. Surviving cells formed colonies, which could be clearly seen under a microscope, then cells were retrieved by cloning disks and plated into the wells of a 24-well plate. In all subsequent experiments, engineered cell lines were cultured in media supplemented with 1 μg/ml puromycin.

### Example 2

### Cell Encapsulation

To facilitate cell loading into hybrid constructs, βTC3 cells and recombinant HepG2 and C2C12 cells were encapsulated in 2% alginate (ISP Inc., San Diego, Calif.) at a density of 7×10⁷ cells/ml of alginate following the general protocol published by Stabler et al (Stabler et al. 2001), except that a poly-L-lysine membrane and a final alginate coating were not applied. This type of encapsulation does not introduce any significant resistance to the transport of cellular nutrients and metabolites. Briefly, cells from confluent monolayer cultures were detached by EDTA-trypsin (Sigma). A sample of the cell suspension was used for cell counting with trypan blue (Sigma); the rest of the suspension was centrifuged for 4 minutes at 110 g, and pellets were mixed with sodium alginate sterilized by filtration through a 0.2-μm syringe filter (Pall Life Sciences, Ann Arbor, Mich.). Alginate beads of approximately 700 μm diameter were formed by passing the cell-alginate suspension through an electrostatic bead generator (Nisco Engineering Inc., Zurich, Switzerland) into a 1.1% CaCl₂ solution. Beads were washed twice with culture medium, incubated overnight on an orbital shaker, and loaded in constructs for secretion experiments the following day. To ensure that recombinant HepG2 and C2C12 cells were functional once encapsulated, beads were also cultured in T-flasks on a rocking platform for up to 10 days with medium changes every one to three days, depending on the cell growth rate. At different times, bead samples were withdrawn for histology and viability assessment by trypan blue staining as described in the analytical techniques. As βTC3 cells have been shown to retain their function after alginate encapsulation, T-flask cultures of alginate beads with these cells were not performed.

### Example 3

### Characterization of Insulin Release from Constructs

Each construct was cultured for 20 hours prior to glucose concentration changes to allow insulin to accumulate inside the cell compartment. Two square-waves of glucose were implemented to test the glucose responsiveness of the hybrid construct. Immediately prior to the glucose changes, the culture medium was replaced with fresh, so when an experiment started at t=0, the surrounding medium was insulin-free and contained 25 mM of glucose. Following incubation for 2 hours in this medium, 2.0 g of glucose was directly added to the beaker to increase the glucose concentration to 220 mM. This glucose concentration is necessary to achieve transformation of the con A-based material from gel to sol (Cheng et al. 2004). For the glucose step down 2 hours later, the medium was completely replaced with fresh culture medium containing 25 mM glucose, and the cycle was repeated once more. Although higher than physiologic, use of these glucose concentrations was compatible with the objectives of this in vitro study. Samples were collected every 30 minutes during experiments, and the same amount of culture medium was added back to maintain constant volume inside the beaker. All samples were stored at −20° C. for later insulin assay. At the end of the experiment, the constructs were disassembled, and the alginate-encapsulated cells were collected to assess cell viability.

### Example 4

### Analytical Techniques

a. Cell Viability

To measure the viability of alginate-encapsulated cells, beads were dissolved in a 2.2% sodium citrate solution, and the resulting cell suspension was centrifuged for 3 minutes at 110 g. Pelleted cells were re-suspended in culture medium and mixed with trypan blue (Sigma) at 1:1 volume ratio. Cell viability in the final suspension was determined on a hemocytometer under a microscope by trypan blue exclusion.

b. Insulin Assay

Insulin secreted by βTC3 and HepG2 cells was assayed by rat insulin radioimmunoassay (Linco Research, St. Charles, Mich., USA). The antibody used in the assay had a 100% reactivity against mouse and human insulin and a 69% cross-reactivity against human proinsulin, hence the immunoreactive insulin assayed in the βTC3 and HepG2 cultures was an aggregate of secreted insulin and any proinsulin. As myoblasts are particularly promising for an autologous cell therapy approach, insulin secreted by recombinant C2C12 cells was assayed by a human-specific insulin radioimmunoassay, which had only a 0.02% cross-reactivity to human proinsulin. This ensured that the assayed immunoreactive insulin in C2C12 cultures was solely mature insulin.

c. Histology

For histology, alginate beads were fixed with a 3% glutaraldehyde solution, then embedded in paraffin blocks. Blocks were later sectioned by a microtome, and the obtained slides were stained with hematoxylin/eosin (H/E).

### Example 5

### Construct with βTC3 Cells

Results with constructs consisting of βTC3 cells and glucose-responsive material (experiment) or culture medium (control) are shown in FIG. 3. βTC3 cells are glucose-responsive but hypersensitive, as they express the GLUT-1 instead of the GLUT-2 glucose transporter and hexokinase instead of glucokinase. Thus, the cells secreted insulin constitutively under the 25-220 mM glucose used in this experiment. The average insulin release rate over each indicated time period was calculated from the slope of the least squares fit of the released insulin concentration vs. time data; each rate was normalized to the corresponding low glucose release rate during the 0-2 hour period, which was set equal to 1. For the experimental construct, the insulin release rates changed with the step changes of glucose: the rates during high glucose were, on average, 2- to 3-fold higher than those during the low glucose periods. On the other hand, insulin release rates from the control construct did not respond to changes in the surrounding glucose level. These results demonstrate that the presence of the con A-based material makes insulin release from the construct glucose-responsive. This occurs while the cells within are glucose-unresponsive, as is also the release of insulin from the control construct containing inert material.

### Example 6

### Construct with Genetically Engineered Non-β Cells

One embodiment provides an autologous cell therapy approach for insulin-dependent diabetes and is based on non-β cells that can be retrieved as a biopsy and genetically engineered to secrete insulin. Such cells include hepatocytes, retrieved as a biopsy from the liver, and myoblasts, retrieved as a biopsy from muscle tissue. Human HepG2 hepatomas and mouse C2C12 myoblasts were genetically engineered for constitutive expression of insulin, encapsulated in calcium alginate beads and associated with glucose-responsive material (experiment) or with calcium alginate (control) in the disk-shaped device shown in FIG. 1. The construct responses to step changes in glucose concentration were then evaluated.

### Example 7

### Construct with HepG2 Cells

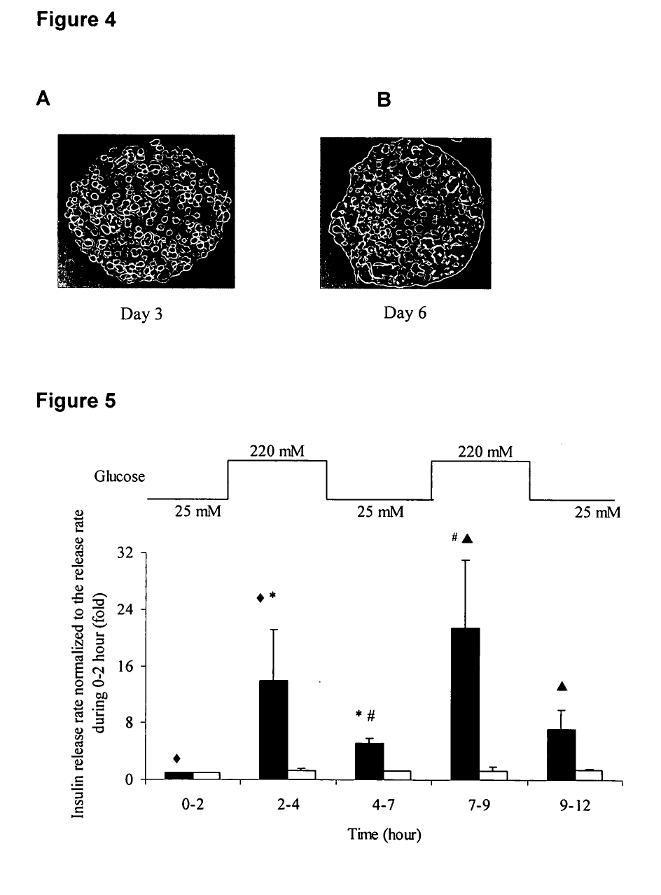

HepG2 cells were genetically engineered by chemical transfection with the CMV/Furin-B10 PPI cDNA/Puro^{R} plasmid shown in FIG. 2A. Clones that survived under the selection pressure secreted insulin constitutively at a rate of 9.5±2.3 μU/(hour·10⁵ cells). This is approximately 6-fold lower than the specific insulin secretion rate of 60-65 μU/(hour·10⁵ cells) reported for βTC3 monolayers at 25 mM glucose. To facilitate loading, cells were encapsulated at a density of 7×10⁷ cells/ml alginate prior to incorporation in the hybrid construct. Histology sections of alginate encapsulated cells propagated in T-flasks on an orbital shaker are shown for two time points in FIG. 4. Cells proliferated in the alginate beads, which remained stable until at least day 6. No bead instabilities, therefore, occurred in hybrid constructs during secretion experiments, which were terminated by day 3 after bead preparation. Incidentally, as no poly-L-lysine coating was applied, excessive cell growth did lead to bead instability and cells escaping from beads by day 9 post-encapsulation (results not shown). Additionally, trypsinization and encapsulation had no short-term effect on insulin secretion from HepG2 cells, as the per-cell secretion rate from calcium alginate-entrapped cells two days post-encapsulation was the same as the secretion rate from monolayer cultures.

Results from experimental and control constructs with HepG2 cells are shown in FIG. 5. In the control construct, insulin-release rates stayed at the same level through the two glucose square-waves in the surrounding medium (P>0.1 between high and low glucose periods). On the other hand, in the experimental construct, insulin-release rates responded to changes in the surrounding glucose concentration. In fact, the average rates showed an 8-fold difference between high and low glucose. Contrary to the experiment with βTC3 cells, in the experiment with recombinant HepG2 cells and the glucose-responsive material the insulin-release rate during a 25 mM glucose period was higher than the rate during the prior 25 mM glucose period. For example, the rate during the 4-7 hour period was higher than that during the 0-2 hour period (p<0.001). This appeared to be due to degradation and loss of con A-glycogen material exaggerated by the presence of the high FBS concentration in the HepG2 culture medium. This possibility was further investigated in experiments with C2C12 cells (see below). Even though the material was not able to completely revert to the original gel state after lowering the glucose level, the insulin release rate during a 25 mM glucose period was always statistically lower than that during the previous 220 mM glucose period (e.g. p<0.05 between the rates for hours 2-4 and 4-7).

### Example 8

### Construct with C2C12 Cells

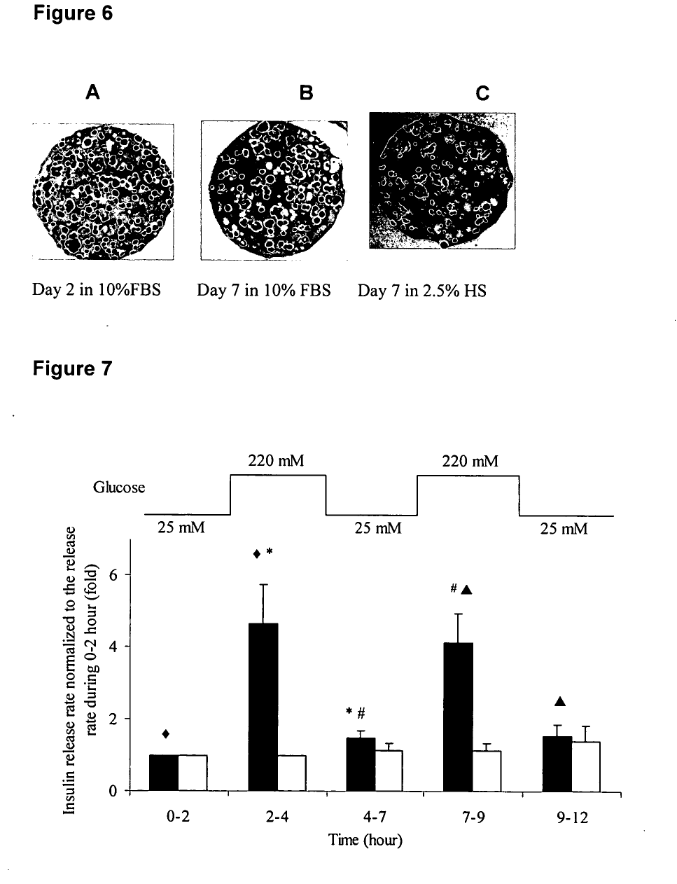

Stable C2C12 clones isolated under antibiotic selection secreted recombinant insulin constitutively when cultured as undifferentiated myoblasts or as differentiated myotubes in monolayer cultures. The insulin-secretion rate from undifferentiated C2C12 myoblasts was 17.5±3.4 μU/hr-10⁵ cells, whereas C2C12 myotubes secreted insulin at a rate of 43.4±9.5 μU/hr-10⁵ cells. This is higher than the insulin secretion rate from recombinant HepG2 cells but lower than the insulin secretion rate from βTC3 monolayers. The higher insulin-secretion rates from myotubes relative to undifferentiated C2C12 cells is consistent with literature reports. C2C12 were encapsulated in alginate at a density of 7×10⁷ cells/ml alginate and were propagated in either FBS or HS-supplemented culture media for 10 days. Histology sections of beads at different time points are shown in FIG. 6. Unlike HepG2 cells, C2C12 cells did not appear to proliferate after encapsulation, especially in HS-supplemented medium. C2C12 cells are reportedly unable to differentiate when plated on alginate sheets and remained undifferentiated in alginate beads in this study, too. Alginate entrapped C2C12 cells secreted insulin post-encapsulation in both types of culture medium used, and the per-cell secretion rate was 9.1±1.7 μU/hr-10⁵ cells, lower than rates measured in monolayer cultures.

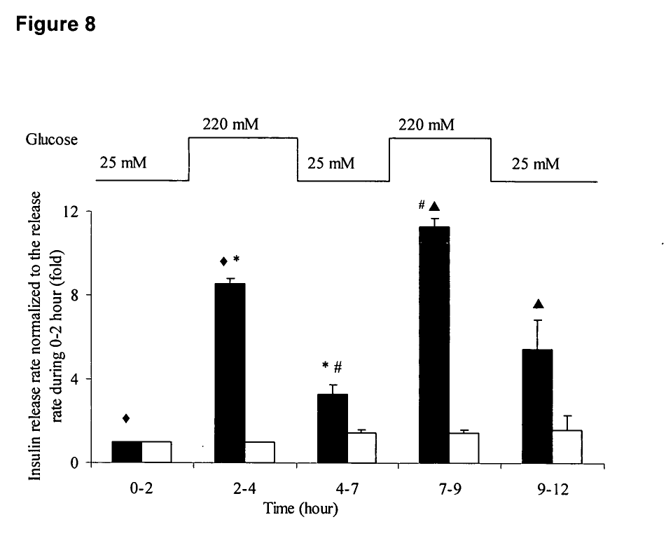

Although C2C12 cells were not able to differentiate in alginate via exposure to low-serum medium, construct experiments were still performed with both 10% FBS and 2.5% HS-supplemented media to further investigate whether a high FBS concentration caused the con A-glycogen degradation observed with HepG2-containing constructs. Insulin-release profiles from the C2C12-based constructs are shown in FIGS. 7 and 8. Insulin release rates from control constructs were at the same level in high and low glucose with both types of serum. On the other hand, the insulin release rates from experimental constructs responded to changes in glucose concentration, but the degree of induction differed with type of culture medium. With 2.5% HS-supplemented medium, the insulin release rates under low glucose remained constant (P>0.3 between values at 4-7 hours and 9-12 hours), and the insulin release rate under high glucose was, on average, 5-fold higher than the corresponding low glucose rate (FIG. 7). This result correlated well with the experiments with βTC3 cells (FIG. 3), in which the culture medium contained 2.5% FBS and 15% HS. With the 10% FBS-supplemented medium, results (FIG. 8) were similar to those obtained with the HepG2-containing constructs, in which the same type of medium was used (FIG. 5). Insulin release rates during the high glucose periods were all higher than that the rates during the low glucose periods, which demonstrated that the release of insulin from the experimental construct was glucose-dependent. However, the insulin release rates during the 25 mM glucose periods increased with time in the experiment: the rate during the 4-7 hour period was 3.3-fold higher than the rate during the 0-2 hour period; and the rate during the 9-12 hour period was 5.5-fold higher than that during the first low glucose period. Furthermore, insoluble particulate matter was observed in the containers at the end of the 20 hour incubation period prior to the glucose concentration step changes only when the high FBS medium was used with both the HepG2 and C2C12 cells as insulin source. Thus, it appears that the high concentration of FBS exaggerated the degradation and loss of con A-based material from the membrane sandwich.

### Example 9

### Cell Viability

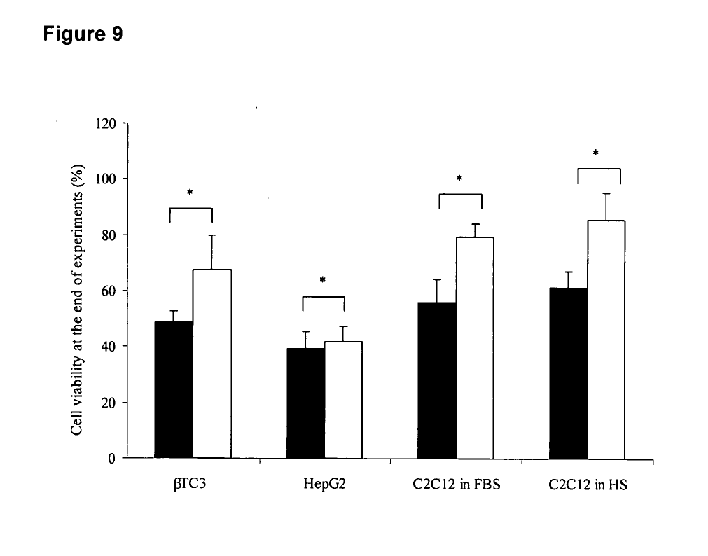

The viabilities of different cell lines in experimental and control constructs are shown in FIG. 9. In control constructs, βTC3 and C2C12 cells maintained viabilities above 60% at the end of 32 hour-long tests. The viability of HepG2 cells in control constructs dropped to below 50% by the end of tests, which suggested that HepG2 cells were more sensitive to the environment inside constructs. Cell viabilities in experimental constructs were statistically lower than those in the corresponding control constructs.

### Example 10

### Pegylated Con A Based Glucose Responsive Material

Con A was modified with PEG using Acrl-PEG-NHS (Molecular weight of 3,400, Nektar Therapeutic, Huntsville, Ala.), which has two functional groups: the NHS group (N-hydroxylsuccinimide) is reactive towards lysine groups and is widely used for protein PEGylation; the Acrl (acrylate) group offers the possibility of vinyl polymerization, which can be used to tether con A to polymer chains if needed. Such tethering was not implemented in the present study. Con A (Amersham Biosciences, Piscatway, N.J.) was first dissolved in PBS at 15% (w/v). Weighted Acrl-PEG-NHS was then slowly added to the con A solution at molar ratios of Acrl-PEG-NHS to con A of 5.0:1.0 and 2.5:1.0. The reaction was carried out in an ice bath for 30 minutes. PEGylated con A tended to form white precipitates and compromised its ability to form glucose-responsive material with glycogen when left too long in the solution. Hence, glucose-responsive materials were prepared immediately after the reaction. Briefly, a con A solution with or without the step of PEGylation was mixed with a 30% glycogen (Sigma) solution, which was prepared by dissolving glycogen in PBS at 60% w/v followed by mixing with a 0.2 M NaIO₄ (Sigma) solution at 1:1 volume ratio for 24 hours in the dark. The viscous gel was formed immediately after stirring the mixtures of glycogen and con A and was left at room temperature 24 hours followed by 1 hour immersion in 1 mg/ml NaBH₄ (Sigma) at 4° C. Finally, the gel was washed at least three times with PBS buffer and stored in PBS at 4° C. for later experiments.

The slope ratio of PEG: con A (molar ratio 5) to unmodified con A is 62.7±9.2%, which indicated that an average of four to five lysine groups per con A molecule were reacted with PEG molecules after the reaction (13 primary amine groups per con A molecule x(1-62.7%)=4.8). Similar calculation was done with PEG: con A (molar ratio 2.5) and a slope ratio of 82.7±3.1 was obtained, which indicated that about two to three PEG molecules is attached to one con A molecules (2.2). The notation of “PEG 2.5-Con A” and “PEG 5-con A” will be used for PEG/con A molar ratio of 2.5 and 5, respectively.

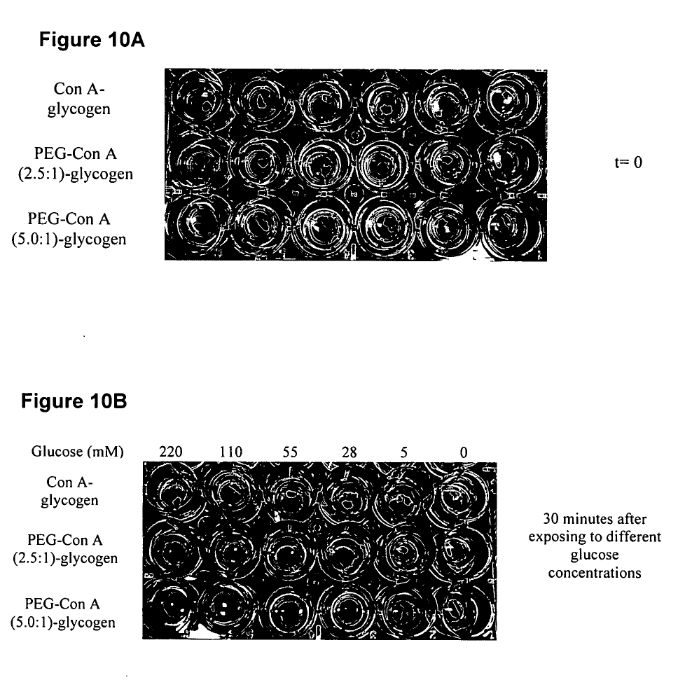

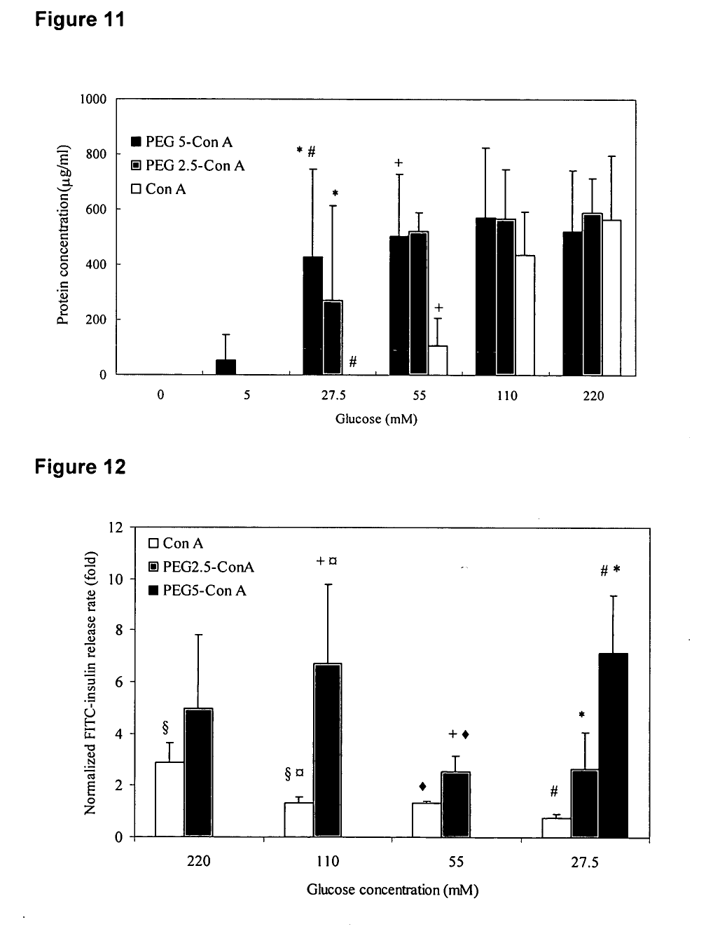

Shortly after mixing the glycogen and PEG-con A solutions, a viscous gel was formed. The gel formed with PEGylated con A was softer and less opaque compared to the gel formed with unmodified con A. Within 30 minutes after exposing the gels to different glucose concentrations, the PEG 5-con A-glycogen material remained as gel at 5 mM glucose but turned into a sol at 27.5 mM glucose, whereas the PEG 2.5-con A-glycogen material was partially converted to sol at 55 mM glucose and completely converted to sol at 110 mM. Most of the unmodified con A-glycogen material remained in the gel state at glucose concentrations tested (FIG. 10). These results thus indicated that the glucose-responsiveness of the con A-based material can be improved by using PEGylated con A. PEGylation of con A is expected to increase the molecular weight of con A molecules, and hence decrease its permeability through membranes. However, 6 hours after exposing the gel to different glucose concentrations, higher protein concentrations were detected outside the 0.02-μm pore size tissue culture inserts at 0-55 mM glucose when PEGylated con A was used relative to unmodified con A (FIG. 11).

Experiments with FITC-labeled insulin were carried out at room temperature and 20 ml PBS with 0.2 mg/ml BSA as the surrounding solution. The construct was incubated in glucose-free PBS for 2 hours, during which time the material was in the gel state. Then, glucose was added into the beaker to increase the glucose concentration and the experiments were continued for another 2 hours. The FITC-insulin release rates during glucose-free and high-glucose periods were calculated by the slopes of the least squares fit of the released FITC-insulin concentration vs. time data; rates at high glucose periods were normalized to the corresponding glucose-free release rate during the 0-2 hour period. The results from three independent experiments were shown in FIG. 12. The change of FITC-insulin release rates from 0 to 27 mM glucose was most pronounced with PEG 5-con A-glycogen material, indicating that material formed with PEG 5-con A-glycogen can be used to control insulin release at glucose concentrations close to the physiologic range.

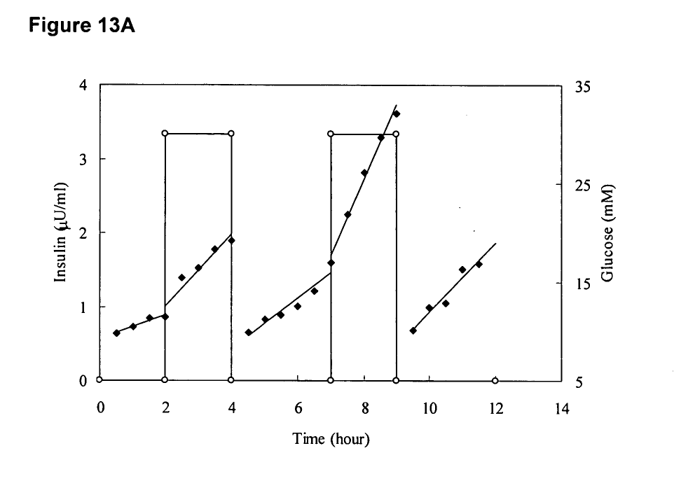

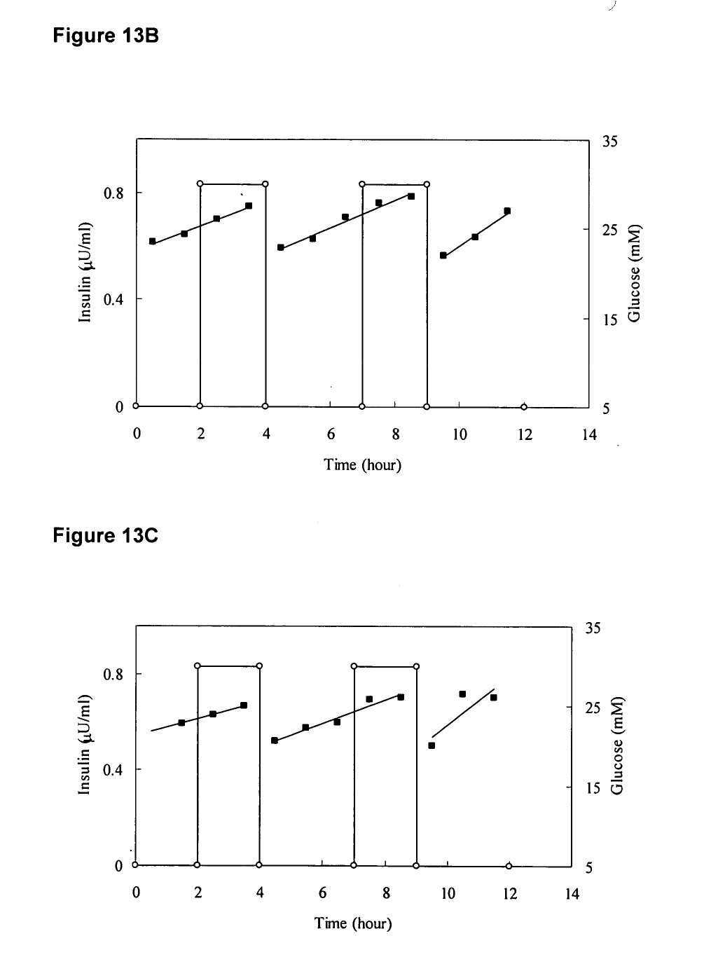

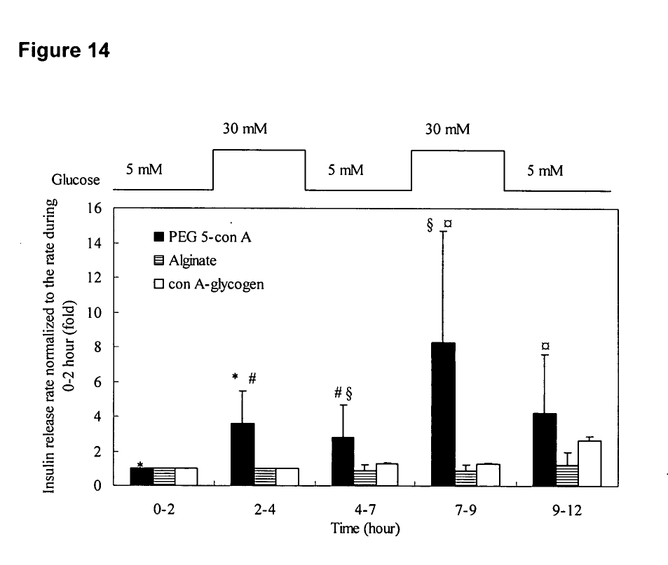

As PEG5-con A-glycogen exhibited glucose responsiveness closest to the physiologic range, it was the material used in construct experiments with cells. FIGS. 13A, 13B, and 13C show a typical insulin-release profile from the cell-material hybrid construct containing three different types of materials: PEG5-con A-glycogen, alginate, and con A-glycogen, under glucose square-wave changes from 5 to 30 to 5 mM. The average insulin release rate in each glucose concentration was calculated from the slope of the least squares fit of the released insulin concentration vs. time data; each rate was normalized to the corresponding low-glucose release rate during the 0-2 hour period, which was set equal to 1. The results from three independent experiments are shown in FIG. 14. Statistical differences were evaluated by the Student's t-test and were considered significant at p<0.05. Constructs containing alginate or unmodified con A-glycogen material acted as continuously insulin-release devices, as they did not respond to changes in the surrounding glucose concentration. On the other hand, insulin release rates from the construct containing PEG 5-con A-glycogen changed with the implemented step changes of glucose concentration: the rates during high glucose were, on average, 2- to 3-fold higher than those during the low glucose periods. However, the PEG 5-con A-based material became more fluid after 32 hours of experiment.

1. A device comprising:
a compartment comprising an insulin producing cell; and
a glucose-responsive material adjacent to the compartment, wherein insulin permeability of the glucose-responsive material changes in response to changes in glucose concentration of fluid in contact with the glucose-responsive material.

2. The device of claim 1, wherein the glucose-responsive material and the compartment are separated by a membrane permeable to insulin produced by the cell.

3. The device of claim 1, wherein the insulin producing cell is a recombinant cell.

4. The device of claim 3, wherein the recombinant cell is not a P-cell.

5. The device of claim 3, wherein the recombinant cell is selected from the group consisting of a hepatocyte, myoblast, embryonic stem cell, adult stem cell, adipocyte, bone marrow cell, umbilical chord blood cell, and fibroblast.

6. The device of claim 1, wherein the glucose-responsive material comprises concanavalin A.

7. The device of claim 6, wherein the glucose-responsive material further comprises a polysacchride.

8. The device of claim 7. wherein the polysaccharide is selected from the group consisting of glycogen, dextran, and synthetic polysaccharides.

9. The device of claim 6, wherein the glucose-responsive material becomes a sol when contacted with a fluid having a glucose concentration of about 25 mM or more.

10. The device of claim 6, wherein the concanavalin A is pegylated.

11. The device of claim 1, wherein the device is configured for implantation into a host.

12. The device of claim 10, wherein the insulin producing cell is derived from the host or obtained from the host.

13. A method for regulating glucose levels of a host comprising:
implanting the device of claim 1 into the host.

14. An implant comprising:
a compartment comprising a continuous source of insulin; and
a glucose-responsive material adjacent to the insulin compartment, wherein insulin permeability of the glucose-responsive material changes in response to changes in glucose concentration of fluid in contact with the glucose-responsive material.

15. The implant of claim 13, wherein the continuous source of insulin comprises one or more insulin producing mammalian cells.

16. The implant of claim 14, wherein the one or more insulin producing mammalian cells are not β-cells.

17. The implant of claim 13, wherein the glucose-responsive material becomes a sol when contacted with a fluid having a glucose concentration of about 25 mM or more.

18. A hydrogel comprising:
pegylated concanvalin A comprising a polyethylene glycol:concanavalin A molar ratio effective to solubilize the hydrogel when the hydrogel is contacted with a fluid comprising mammalian physiological levels of glucose; and
a polysaccharide.

19. The hydrogel of claim 18, wherein the polyethylene glycol:concanavalin A molar ratio is from about 2.5 to about 5.

20. The hydrogel of claim 18, wherein the hydrogel becomes a sol when contacted with a fluid having a glucose concentration of about 25 mM or more.

21. The hydrogel of claim 18, wherein the polysaccharide comprises glycogen.
